(19) United States
(12) Patent Application Publication
(10) Pub. No.: US 2013/0023058 A1
(43) Pub. Date: Jan. 24, 2013

(54) METHOD AND APPARATUS FOR SELECTING A PRODUCT

(71) Applicants: Christofer TOUMAZOU, London (GB); Calvin Sim, Kowloon (HK)

(72) Inventors: Christofer TOUMAZOU, London (GB); Calvin Sim, Kowloon (HK)

(73) Assignee: Gene Onyx Limited, Roadtown (VG)

(21) Appl. No.: 13/431,514

(22) Filed: Mar. 27, 2012

(30) Foreign Application Priority Data
Jul. 19, 2011 (GB) 1112421.1

(51) Int. Cl. G06Q 50/22; G01N 33/53

(52) U.S. Cl. 436/501; 705/2

(57) ABSTRACT

A method of selecting a product suited to an individual. The method includes: identifying a product category from a predefined set of product categories, each product category being associated with a set of products; on the basis of said selected product category, selecting a genetic testing cartridge type from a set of available cartridge types, each cartridge type being configured to perform a genotype profiling test on a polynucleotide sample; collecting a polynucleotide sample from the individual; performing a genotype profiling test on said polynucleotide sample using a genetic testing cartridge of the selected genetic testing cartridge type; and using the result of said test to select a product from within the identified product category.

### TECHNICAL FIELD

The present invention relates to a method and apparatus for selecting a product.

### BACKGROUND

The cosmetic products market is large and diverse with numerous brands, product types and wide ranges of similar products. A recent market research survey has found that in 2010 consumers spent nearly US$53 billion on cosmetics and beauty products.

The array of choices of available cosmetic products may be bewildering and an individual's approach to selecting a product could typically be based on any combination of the following selection criteria: brand, price, packaging aesthetics, indicated usage, prior experience where available and also marketing input from the retail assistant. Often, where there is no prior experience with the product, there is little, if any, guidance tailored to an individual that could be relied upon to indicate the efficacy and allergenic potential of the cosmetic product to that individual.

Consequently, a trial and error method is often used by consumers to assess and purchase cosmetic products. When a purchased product turns out to be unsuitable or ineffective, the consumer will discontinue using the product, and often will also discard it, leading to wastage and consumer frustration. In some more severe cases, and particularly for people with sensitive skin, adverse allergic reactions may occur leading to unsightly reddening, swelling and/or itching, and in extreme cases, may be life threatening.

Currently, manufacturers, marketers and/or vendors of cosmetic products are attempting to address this problem through the use of scientific methods of investigation and analysis of a potential buyer's skin condition. When this is performed “on-site” (e.g. in the shop with the potential buyer present), the results and conclusions are used to offer recommendations on appropriate products and/or remedies. However, on-site investigations and analysis currently only address the physiological expression or symptom of traits of the individual and does not shed any insight on the underlying genetic cause of the predisposition or condition.

“Off-site” testing (e.g. away from the shop, when the potential buyer is no longer present) allows for investigations based on genotyping of the individual and these tests are now available commercially. However, the need to send their genetic material to a remote site can increase a worried consumer's concerns on the storage and tracking of individual genetic material. Moreover, given that the successful sale of new cosmetic products is often driven by meeting a mixture of human needs and desires in a timely fashion, it is important that this process of product guidance is performed within a short timeframe and while the potential customer's interest is sustained. In discerning and accurately responding to a customer's needs, having a sales assistant who is able to guide and respond in a personable, face-to-face manner also significantly increases the likelihood of achieving customer satisfaction. In this regard, existing commercial products that require genetic tests to be performed off-site are unable to have or sustain a personal engagement with the customer right through the entire process.

GB 2389424 discloses a rapid genotyping technology which is capable of performing single nucleotide polymorphisms (SNPs) genotyping through electro-chemical means in the order of minutes rather than days with traditional optical PCR approaches.

### SUMMARY

According to a first aspect of the invention, there is provided a method of selecting a metabolisable product suited to an individual, the method comprising:
providing a database mapping each of a set of metabolisable products to one or more single nucleotide polymorphisms (SNPs) associated with the metabolisation of the metabolisable product;
making the database available at a point-of-sale locale together with cartridges configured using polynucleotide probes to perform targeted tests for the presence of individual SNPs, identified in said database, in customers' polynucleotides; and
periodically adding further said mappings to the database, and making the updated database available to the point-of-sale locales together with disposable cartridges configured using polynucleotide probes to perform targeted tests for the presence of SNPs associated with said further mappings.

The metabolisable product may be one of: a cosmetic, a cosmeceutical, a nutricosmetic, nutritional supplement, a food group, and an agricultural product.

The cartridges may be configured to accept a customer's polynucleotide sample from a swab or saliva sample, and may be disposable cartridges.

The polynucleotide may be DNA, RNA or a combination of both.

The products may be products packaged for sale, or may be active ingredients (compounds) that are contained within products packaged for sale.

According to a second aspect of the invention there is provided a method of selecting a product suited to an individual, the method comprising:
identifying a product category from a predefined set of product categories, each product category being associated with a set of products;
on the basis of said selected product category, selecting a genetic testing cartridge type from a set of available cartridge types, each cartridge type being configured to perform a genotype profiling test on a polynucleotide sample;
collecting a polynucleotide sample from the individual;
performing a genotype profiling test on said polynucleotide sample using a genetic testing cartridge of the selected genetic testing cartridge type; and
using the result of said test to select a product from within the identified product category.

The step of using the result of said test to select a product may comprise using the result of said test to select one or more metabolisable compounds from a group of known metabolisable compounds, and then selecting a product from the identified product category that contains at least one of the one or more selected metabolisable compounds.

The one or more metabolisable compounds may be selected by determining the efficacy of the known metabolisable compounds with respect to the metabolism pathway of the individual, and selecting the one or more with the highest efficacy.

The polynucleotide sample may contain DNA, RNA or a combination of both.

Said genotype profiling test may comprise one or more single-nucleotide polymorphism tests.

Said product may be one of: a skin care product, a cosmetic, a cosmeceutical, a nutricosmetic, nutritional supplement, a food group, and an agricultural product.

Said step of using the result of said test to select a product from within the identified product category may comprise performing a lookup in a product database for the identified product category, using as lookup key the test result.

Said step of identifying a product category may comprise identifying a product category to a point of sale terminal using a graphical user interface of the terminal, the point of sale terminal performing the steps of selecting a genetic testing cartridge type and using the results of said test to select a product from within the identified product category, the selection being displayed to a terminal user via said graphical user interface.

The method may comprise deleting all records of the test result from the point of sale terminal following selection of a product.

Said step of collecting a polynucleotide sample from an individual may be performed by the individual or a point of sale representative, the individual or point of sale representative applying the collected sample to a genetic testing cartridge of the selected type.

The method may be carried out at a retail premises.

The method may comprise receiving a questionnaire response from said individual, and using said response in the selection of a genetic testing cartridge type and/or in the selection of a product.

Said genetic testing cartridge of the selected genetic testing cartridge type may be configured to determine the efficacy of the associated product set.

Said step of selecting a product category may comprise:
collecting a polynucleotide sample from the individual;
using a genetic testing cartridge to perform a genotype profiling test and using the result of the test to identify a predisposition of the individual to each of one or more conditions; and
for a given one or combination of the identified predispositions, choosing a product category that can potentially treat the associated condition(s).

The method may comprise using additional information obtained for the individual, for example lifestyle information, in the identification of the condition predispositions and/or the choosing of the product category.

Said genetic testing cartridge may be a disposable single-use cartridge.

According to a third aspect of the invention there is provided a point of sale terminal comprising:
access to memory that may be local or remote to the terminal, in which memory there is stored a first database containing a set of product categories and a set of genetic testing cartridge types mapped to the product categories, and a second database containing, for each product category, a set of available products and a set of genotype profiling outcomes mapped to the available products;
an interface for coupling the point of sale terminal to a genetic testing cartridge in order to receive test results from the cartridge;
a graphical user interface for receiving from a terminal user an identification of a product category and for displaying to the user a selected product; and
a processing entity for using the received identification of a product category to lookup said first database to select a genetic testing cartridge type, for receiving a test result via said interface, for using the received test result as a lookup in said second database to select a product, and for causing the graphical user interface to display the selected product to a user.

The point of sale terminal may comprise a holder for retaining a set of genetic testing cartridges belonging to said cartridge types.

Said processing entity may be configured to control said holder to deliver from the holder a test cartridge of said selected test cartridge type, and said holder may deliver said test cartridge to a position in which it is connected to said interface.

Said processing means may be configured to allow said second database to be updated to reflect available products within a retail premises within which the terminal is located.

Said memory may store a third database containing a set of condition predispositions and a set of genotype profiling outcomes mapped to said condition predispositions, and said processing entity may be configured to receive a test result from a genetic testing cartridge, and use the received result as a lookup to said third database to identify one or more condition predispositions.

The processing entity may be configured to use the received result as a lookup to said third database in combination with other data obtained for the individual, such as lifestyle information.

Said processing entity may be further configured to display the or each condition predisposition via the graphical user interface, thereby facilitating said selection of a product category suited to treating a condition to which the individual is predisposed.

Said memory may store a further database containing a set of known metabolisable compounds found in the products in the second database, and said processing entity is configured to receive a user's test result from a genetic testing cartridge, and use the received result to determine the efficacy of one or more of said known metabolisable compounds with respect to the metabolism pathway of the user, then to select a product from the second database based on the determined metabolisable compound efficacies for the user.

Each product within the second database may be one of: a skin care product, a cosmetic, a cosmeceutical, a nutricosmetic, nutritional supplement, a food group, and an agricultural product.

The genetic testing cartridge may be a disposable single-use cartridge.

### BRIEF DESCRIPTION OF THE DRAWINGS

FIG. 1 shows a typical service cycle of a customer according to an embodiment of the current invention;

FIG. 2 shows a flow diagram outlining the steps of a method to provide guidance to increase the likelihood of a particular product being suitable for an individual;

FIG. 3 shows a graphical depiction of a Recommendations Engine interfacing with a Test Cartridge;

FIG. 4 shows a block diagram of a recommendations engine according to an embodiment of the invention; and

FIG. 5 shows a block diagram of a Test Cartridge for use in rapid genotyping.

### DETAILED DESCRIPTION

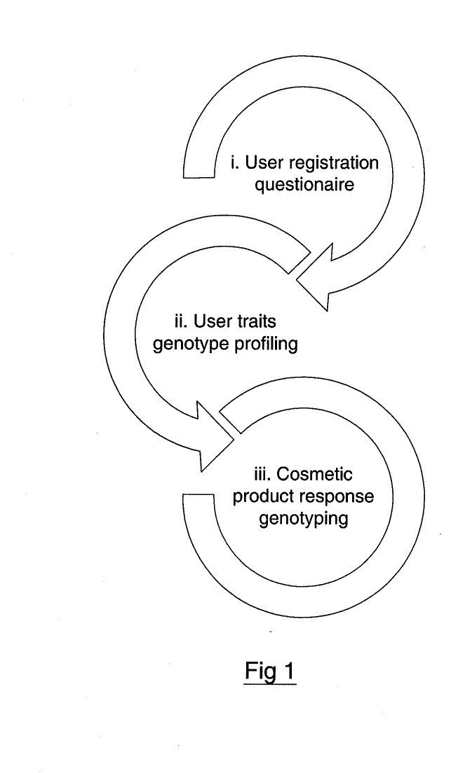

FIG. 1 shows the typical service cycle of a customer according to an embodiment of the current invention. The main components of the cycle are:
i. user registration in which the customer's information is captured through a user questionnaire and can be uploaded to the user database in a central database, such as on an Internet cloud server;
ii. initial genotype profiling to determine the key traits of the customer, such as the customer's propensity to aging, inflammation, etc; and
iii. the customer's response to a set of cosmetic products is predicted through the further capture and analysis of the customer's genotype by profiling the customer against a set of known SNP mutations that characterise the customer's response to specific chemical compounds that exist in the cosmetic product in terms of efficacy and allergenic response. This final step may be repeated each time the customer would like to evaluate the suitability of a recommended cosmetic product.

Taking cosmetic products that are targeted for improving a user's skin as an example, in terms of skin health, there are a number of key pillars that support healthy skin. Factors that affect healthy skin may generally be grouped according to these pillars. The key pillars are:
1. neutralisation of reactive oxygen species;
2. epidermal immune defense;
3. collagen reinforcement;
4. moisture retention/hydration;
5. nourishment and oxygenation; and
6. detoxification.

For each pillar, the factors that effect skin health may include both phenotypical (e.g. environmental) as well as genotypical (i.e. genetic) contributions.

The expressed factors responsible for the phenotypical characteristic of an individual user for a particular pillar of skin health could be determined from responses to a customer questionnaire. In one instance, the questionnaire could be presented as a collection of statements to which the respondent has to rate on a scale, such as from 0 to 5, to what extent they would agree with each statement. The statements may be carefully and deliberately constructed in consultation with expert advice to provide an accurate assessment of an individual's skin health in relation to one or more key pillars.

To ensure consistency, the impact of each of the statements towards each pillar of skin health may be quantified and collated in a matrix, as illustrated in Table 1.

It is then be possible to mathematically model and derive a quantitative assessment for each pillar of an individual's skin health based on the individual's questionnaire responses. The individual's skin health may then be characterised numerically as {Qₚ₁, Qₚ₂, . . . , Qₚ₆, Qₐ₁}, where Qₚₙ and Qₐ₁ are the aggregate scores for each pillar of skin health and allergenic response respectively. A score of zero would indicate ideal healthy skin while any score greater than zero would indicate skin health deficiency.

The next stage of the service cycle is to build up a genotypical profile of the individual similar to the phenotypical profile generated using the customer questionnaire. However, instead of a score based on the individual's responses to specific statements, the score is determined by the genotype of the individual with respect to a set of key genes that regulate skin health. For each of the key genes to be included in the genotyping procedure, its impact to each of the key pillars of skin health is pre-analysed and tabulated within a matrix. For example, the gene MMP1 may have strong impact on collagen reinforcement, some impact on nourishment and oxygenation but minimal impact on skin hydration. For other key genes, their impact on the different pillars will of course be different. The differences are quantitatively related using a matrix. An example of the matrix is shown in Table 2.

The values in the matrix may be stored on the Internet at a cloud server and would only be updated infrequently so as to maximise the level of consistency of the data.

The individual's profile based on his or her genotype for each pillar of skin health may be calculated based on the pre-computed matrix and his/her genotype (including heterozygosity). In this manner, the individual's skin for each of key pillars based on his or her genotype may be numerically characterised as {Gₚ₁, Gₚ₂, . . . , Gₚ₆, Gₐ₁}, where Gₚₙ and Gₐ₁ are the aggregate scores for each pillar of skin health and allergenic response respectively. A score of zero would indicate ideal healthy skin while any score greater than zero would indicate a level of skin health deficiency.

In view of the number of cosmetic products that would match each selection of SNPs, there are a number of ways in which different brands and retailers of cosmetic products will be able to test their customer using their genetic make-up to determine their product's suitability. The customer's genetic information is extracted, for example by a cheek swab, saliva sample, blood test or similar, and then analysed and mapped to the expected phenotype characteristics that those SNPs code for. These steps are carried out in-situ, such that none of the customer's genetic information is transmitted or stored. Rather than providing the genetic information directly to the customer, the outcome of the test is an analysis of the suitability of the customer to a recommended cosmetic product. For example, the customer may be told that he or she has a predisposition for dry skin, and so should consider using skin creams. Consequently, the same set of genetic information may be repeatedly tested so as to ascertain the customer's suitability to different cosmetic brands and products within an available set of skin creams.

It is envisioned that the primary focus of this system is in matching the efficacy and allergenic response of specific skin-care/cosmetic products that are targeted towards improving/augmenting skin health to specific individuals. However, it could equally be possible that such a system is used purely for identifying possible allergenic risks to specific individuals for cosmetic products solely for aesthetic functionality, or for cosmeceuticals, nutricosmetics, nutritional supplements, food groups and/or agricultural products.

Most cosmetic/skin-care products comprise one or more active compounds which are responsible for the target and/or marketed function of the product, as well as supporting bulk compounds. The active compound(s) in a cosmetic product typically target one or more of the pillars of skin health described above in terms of augmenting the intrinsic capabilities of the individual. Information of the use of such a compound as the active ingredient in a dermatological product can be obtained empirically or is available from the literature provided with the cosmetic product. Furthermore, for each compound (both active and bulk), the relative risk of inflammation due to that compound can be quantified with results obtained either empirically through laboratory testing or existing published results.

Consequently, a matrix estimating the quantitative effect of each compound/reagent to each of the pillars of skin health and their associated risk of inflammation can be collated, an example of which is shown in Table 3.

In the example in Table 3, compounds NIA-114, Azathioprine, aaa and bbb are active compounds which would affect skin health while compounds ccc and ddd would be bulk compounds which have little effect on skin health but may still provoke an allergenic response. With such a matrix, it is possible to mathematically model and derive a formula that can quantitatively estimate, and provide a corresponding numerical score, for the efficacy of any skin-care/cosmetic product towards achieving one or more pillars of skin-care health as an aggregate of its compounds, so long as all the constituent compounds and their relative concentration to a standardised volume is known. Similarly, it would be possible to model the product's allergenic response impact for any skin-care/cosmetic product given similar criteria and inputs.

In one instance, the aggregate effect of the product towards p1 may be modelled by:

Eₚₙ=Σγᵣ_{ₙ}pᵣ_{ₙ} (1)

and the aggregate allergenic response may be modelled by:

α=Σγᵣ_{ₙ}aᵣ_{ₙ} (2)

For example, a skin-care product targeted at neutralising free radicals (p1) may comprise compounds r1, r2, r5 and r6 with standardised concentrations of 0.05, 0.1, 0.4 and 0.7 respectively. The impact of the product towards p1 would therefore be estimated as:

Eₚ₁=0.05×0.8+0.1×0.3=0.07

Similarly, the allergenic response of the product would be estimated as:

αₐ₁=0.05×0.1+0.1×0.4+0.4×0.1+0.7×0.2=0.225

For each product, it is possible to quantitatively characterise its effectiveness towards each pillar of skin health with {Eₚ₁, Eₚ₂, . . . , Eₚ₆, Eₐ₁}, where Eₚₙ and Eₐ₁ are the aggregate scores for each pillar of skin health and allergenic response respectively. A score of zero would indicate minimum effectiveness to a particular deficiency while any score greater than zero would quantitatively indicate the level of effectiveness.

Using such an approach, each skin-care product within the recommendation system can be scored and ranked in order for each pillar of skin-health and/or potential allergenic risk. Given the user's skin health characteristic has been previously determined through the user questionnaire and genotype profiling, the list of most relevant skin-care or cosmetic product(s) that is specific to the individual may be determined by minimising the following expression:

Qₚₙ+Gₚₙ−Eₚₙ (3)

In other words, the products that are most relevant to a specific individual are products where the user's characteristics as quantified by Qₚₙ+Gₚₙ are best matched by the level of effectiveness for a particular pillar of skin health, Eₚₙ, of that product.

Ideally, a particular product would work as indicated and provide the desired effect. However, due to the occurrence of SNP mutations that may impact the action/metabolism of the active ingredient(s) in the mechanism pathway, the efficacy of those ingredients may be diminished either completely or partially. It is the objective of the third stage of the service cycle to use genotyping means to evaluate the efficacy of a product to a specific individual and also predict the risk of an allergenic response to the product. The regulation of the effect of a particular product due to mutations in the genotype may be characterised by an efficacy coefficient, γₚₙ, such that the actual compensatory effect of the product for a pillar of skin health would be γₚₙEₚₙ. Hence, in reality, the expression (3) above should be modified to:

Qₚₙ+Gₚₙ−γₚₙEₚₙ (4)

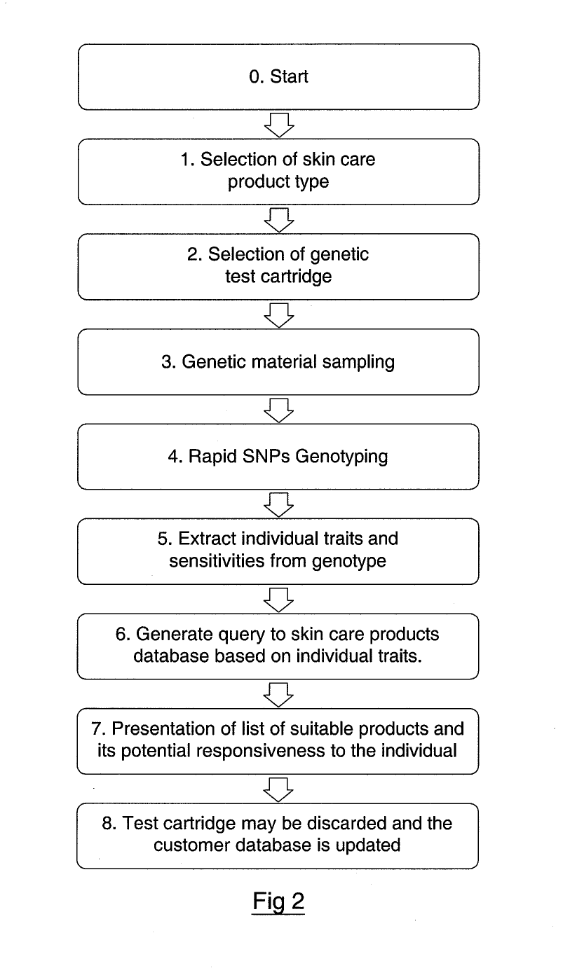

The genotyping performed in the third stage of the service life-cycle is focused on determining the value of γₚₙ for each of the key pillars of skin health. Due to the effect of mutation on the efficacy of the active ingredients in its effectiveness, skin-care products that are expected to work well for a particular skin-health objective for a particular user with a known skin-health characteristic, may still not work as well in reality. By determining the impact of such mutations on the efficacy of a range of potentially suitable products, the suitability for that range of products may be more accurately assessed. FIG. 2 shows a flow diagram that describes a method to provide objective guidance to this process in order to significantly increase the likelihood of a particular skin-care product being suitable for an individual. This is achieved by ascertaining specific traits coded within the genetic material of the individual and matching them to a database of cosmetic products and their constituent ingredients, from which a substantially smaller subset of suitable cosmetic products is presented.

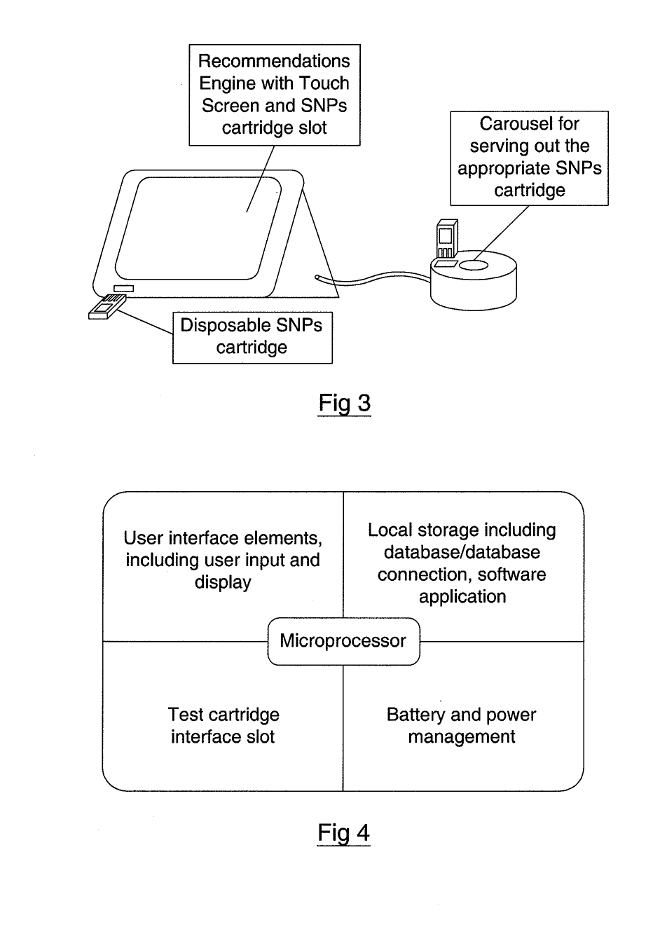

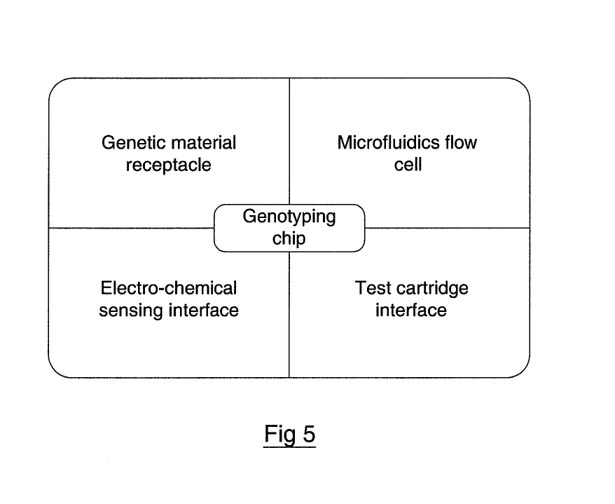

In one embodiment, this method assumes the context of marketing and identifying the appropriate cosmetic product that is suitable for a potential customer in a retail store using custom designed software and hardware device package (the “Recommendations Engine” (RE)). The RE contains a slot for interfacing with the test cartridge (TC), as shown in FIG. 3. The key functional blocks that make up the RE and TC are depicted in FIG. 4 and FIG. 5 respectively. There will typically be a number of different TCs available, with each different TC testing a different selection of SNPs. The TCs will typically be single use and/or disposable cartridges.

Referring again to FIG. 2, in Step 1, the consumer is asked to provide feedback on the type and nature of skin care product of interest (for example an anti-aging mask or a skin-whitening cream). Within a brick-and-mortar retail context, the number of cosmetic products available in a retail store is likely to be limited and within that range of available products, the cosmetic products that have associated genetic suitability test for them may encompass a subset of all the cosmetic products that have associated genetic suitability tests. The point of sale locale is expected to be a shop or retail kiosk.

In Step 2, as there are many SNPs and arrays of SNPs that are known and yet to be discovered that may be relevant in guiding the choice of cosmetic products, and the limitation in the number of SNPs that can be genotyped at one time within a rapid-testing, portable test environment (i.e. the RE), an algorithm is used to suggest the appropriate selection of SNPs to be tested (i.e. the algorithm is used to determine which TC to use). The algorithm makes the selection from the comprehensive set of SNPs tests possible based on the consumer's indicated desires in Step 1, as well as the types of TCs available in the retail store and the selection of available skin-care products in the said retail store. Each TC is coded with a serial number, which could comprise a string of symbols such as an alphanumeric string of which a portion would be indicative of the type of SNPs being tested for in the TC. Based on the said indicative code segment, the traits and properties of the SNPs tested in the TC could be looked up within the TC catalogue database containing information on all possible SNPs and SNP configurations that are available globally. In an advanced embodiment, the selection algorithm may also be influenced by a host of other factors, including but not limited to the previous tests that have been performed (so as to offer a new set of products), the products available on promotion, and the popular products that other customers with similar traits have bought and found useful.

In Step 3, the customer's genetic material, such as from a cheek swap or saliva sample, is obtained and deposited in contact with the chemically active sensor on the selected genetic TC. The genetic material contains a polynucleotide sample comprising DNA, RNA or a combination of both.

In Step 4, the TC is inserted into the RE and an identification and configuration handshake exchange protocol is performed between the RE and TC. Within this handshaking protocol, the type of TC is communicated to the RE through the appropriate TC serial number code segment. Once this handshake is completed, rapid SNPs genotyping is performed in the TC and the results are transmitted electrically to the RE within minutes of the sample being deposited in the TC. The TC will be configured using a polynucleotide probe to perform a targeted test for the presence of one or more specific SNPs.

In Step 5, the results from the genotyping are received by the RE from the TC and translated into specific parameters, each parameter comprising a tuple of the SNP and the result of the SNP test, by querying the TC catalogue database with the relevant TC serial number code segment. An algorithm is then used to map the parameters into characteristic traits and a corresponding score for that particular trait so as to provide an output of a set of tuples of characteristic traits and their corresponding values. The output is referred to as the characteristic traits table. For each type of TC, there would be a characteristic trait table with values derived from the detected genotype of the individual.

In Step 6, the characteristic traits table is used to structure a query in an appropriate database query language (such as SQL) which is then applied to the cosmetic products database to select from the list of cosmetic products available in the retail store the subset list of products to be recommended, based on the customer's genotype information as well as the desired cosmetic product range indicated in Step 1. The query may be submitted to a local database contained within the RE or to a remote database. By transforming the SNPs responses into the characteristics trait table which is then used as filter values for the database query, it is possible to offload the database of all possible cosmetic products to a remote server and perform queries at the remote server without transmitting information that can be used to identify the said individual's genetic makeup. This is desirable as it ensures that there is a high-level of confidence that no individual genetic information is leaked from the system, without having to resort to complex encryption schemes. Onerous warranties from the service provider to the customer regarding the data integrity and security of the customer's genetic information are therefore also not required.

In Step 7, the selected list of recommended products is displayed on the RE and presented to the customer. In an advanced embodiment, a recommendation score that is proportional to the suitability of the product to the individual is displayed against each of the recommended products list. The list may then be sorted according to different parameters, including but not limited to the prices of the recommended products, their brands and their suitability score.

In Step 8, the TC may be removed from the RE and discarded, while “housekeeping” tasks are performed by the RE to update the customer database as well as to reset itself for the next customer/test.

The performance of the genetic test is wholly carried out in situ within the portable device without a need to send the consumer's genetic sample to a remote laboratory. Furthermore, as the test cartridge is single used and disposed of after the test, this provides strong assurance to the consumer that there is no risk of infringement of personal privacy through the storage and tracking of genetic information.

It will be appreciated by the person of skill in the art that various modifications may be made to the above described embodiments without departing from the scope of the present invention.

TABLE 1

| | | Neutralisation of | Epidermal | | | Nourishment | | |
|---|---|---|---|---|---|---|---|---|
| | | reactive oxygen | immune | Collagen | | and | | Inflammation |
| | Statement | species | defense | reinforcement | Hydration | oxygenation | detoxification | risk |
| Statements | index | p1 | p2 | p3 | p4 | p5 | p6 | a1 |
| I need to use moisturiser | s1 | 0.1 | 0.1 | 0.9 | 0.0 | 0.6 | 0.2 | 0.1 |
| products regularly. | | | | | | | | |
| My living environment is | s2 | 0.7 | 0.8 | 0.0 | 0.1 | 0.3 | 0.6 | 0.1 |
| smoke-free. | | | | | | | | |
| I sleep well. | s3 | 0.5 | 0.6 | 0.8 | 0.4 | 0.1 | 0.2 | 0.1 |
| I consume little alcohol. | s4 | 0.5 | 0.2 | 0.7 | 0.8 | 0.9 | 0.4 | 0.3 |
| My schedule is very hectic. | s5 | 0 | 0 | 0 | 0.1 | 0 | 0.1 | 0.1 |
| I suffer from hay fever. | s6 | 0.1 | 0.5 | 0 | 0.1 | 0.2 | 0.4 | 0.8 |

TABLE 2

| | | Neutralisation of | | | | | | |
|---|---|---|---|---|---|---|---|---|
| | | reactive oxygen | Epidermal | Collagen | | Nourishment | | Inflammation |
| | Gene | species | immune defense | reinforcement | Hydration | and oxygenation | detoxification | risk |
| Gene | index | p1 | p2 | p3 | p4 | p5 | p6 | a1 |
| MMP1 | g1 | 0.1 | 0.1 | 0.9 | 0.0 | 0.6 | 0.2 | 0.1 |
| SOD2 | g2 | 0.7 | 0.8 | 0.0 | 0.1 | 0.3 | 0.6 | 0.1 |
| GPX1 | g3 | 0.5 | 0.6 | 0.8 | 0.4 | 0.1 | 0.2 | 0.1 |
| EPHX | g4 | 0.5 | 0.2 | 0.7 | 0.8 | 0.9 | 0.4 | 0.1 |
| TNFa | g5 | 0 | 0 | 0 | 0.1 | 0 | 0.1 | 0.8 |

TABLE 3

| | | Neutralisation of | | | | | | |
|---|---|---|---|---|---|---|---|---|
| | | reactive oxygen | Epidermal | Collagen | | Nourishment | | Inflammation |
| | Compound | species | immune defense | reinforcement | Hydration | and oxygenation | detoxification | risk |
| Compound | index | p1 | p2 | p3 | p4 | p5 | p6 | a1 |
| NIA-114 | r1 | 0.8 | 0.7 | 0.4 | 0.3 | 0.7 | 0.6 | 0.1 |
| Azathioprine | r2 | 0.3 | 0.6 | 0.3 | 0.7 | 0.5 | 0.5 | 0.4 |
| aaa | r3 | 0.5 | 0.6 | 0.8 | 0.4 | 0.1 | 0.2 | 0.5 |
| bbb | r4 | 0.5 | 0.2 | 0.7 | 0.8 | 0.9 | 0.4 | 0.3 |
| ccc | r5 | 0 | 0 | 0 | 0.1 | 0 | 0.1 | 0.1 |
| ddd | r6 | 0 | 0 | 0.1 | 0 | 0 | 0 | 0.2 |

1. A method of selecting a metabolisable product suited to an individual, the method comprising:
providing a database mapping each of a set of metabolisable products to one or more single nucleotide polymorphisms (SNPs) associated with the metabolisation of the metabolisable product;
making the database available at a point-of-sale locale together with cartridges configured using polynucleotide probes to perform targeted tests for the presence of individual SNPs, identified in said database, in customers' polynucleotides; and
periodically adding further said mappings to the database, and making the updated database available to the point-of-sale locales together with disposable cartridges configured using polynucleotide probes to perform targeted tests for the presence of SNPs associated with said further mappings.

2. A method as claimed in claim 1, wherein the metabolisable product is one of: a cosmetic, a cosmeceutical, a nutricosmetic, a nutritional supplement, a food group, and an agricultural product.

3. A method as claimed in claim 1, wherein the cartridges are configured to accept a customer's polynucleotide sample from a swab or saliva sample.

4. A method as claimed in claim 1, wherein the cartridges are disposable cartridges.

5. A method as claimed in claim 1, wherein the polynucleotide is DNA, RNA or a combination of both.

6. A method of selecting a product suited to an individual, the method comprising:
identifying a product category from a predefined set of product categories, each product category being associated with a set of products;
on the basis of said selected product category, selecting a genetic testing cartridge type from a set of available cartridge types, each cartridge type being configured to perform a genotype profiling test on a polynucleotide sample;
collecting a polynucleotide sample from the individual;
performing a genotype profiling test on said polynucleotide sample using a genetic testing cartridge of the selected genetic testing cartridge type; and
using the result of said test to select a product from within the identified product category.

7. A method according to claim 6, wherein the step of using the result of said test to select a product comprises using the result of said test to select one or more metabolisable compounds from a group of known metabolisable compounds, and then selecting a product from the identified product category that contains at least one of the one or more selected metabolisable compounds.

8. A method according to claim 7, wherein the one or more metabolisable compounds are selected by determining the efficacy of the known metabolisable compounds with respect to the metabolism pathway of the individual, and selecting the one or more with the highest efficacy.

9. A method according to claim 6, wherein the polynucleotide sample contains DNA, RNA or a combination of both.

10. A method according to claim 6, wherein said genotype profiling test comprises one or more single-nucleotide polymorphism tests.

11. A method according to claim 6, wherein said product is one of: a skin care product, a cosmetic, a cosmeceutical, a nutricosmetic, nutritional supplement, a food group, and an agricultural product.

12. A method according to claim 6, wherein said step of using the result of said test to select a product from within the identified product category comprises performing a lookup in a product database for the identified product category, using as lookup key the test result.

13. A method according claim 6, wherein said step of identifying a product category comprises identifying a product category to a point of sale terminal using a graphical user interface of the terminal, the point of sale terminal performing the steps of selecting a genetic testing cartridge type and using the results of said test to select a product from within the identified product category, the selection being displayed to a terminal user via said graphical user interface.

14. A method according to claim 13 and comprising deleting all records of the test result from the point of sale terminal following selection of a product.

15. A method according to claim 6, wherein said step of collecting a polynucleotide sample from an individual is performed by the individual or a point of sale representative, the individual or point of sale representative applying the collected sample to a genetic testing cartridge of the selected type.

16. A method according to claim 6, the method being carried out at a retail premises.

17. A method according to claim 6, and comprising receiving a questionnaire response from said individual, and using said response in the selection of a genetic testing cartridge type and/or in the selection of a product.

18. A method according to claim 6, wherein said genetic testing cartridge of the selected genetic testing cartridge type is configured to determine the efficacy of the associated product set.

19. A method according to claim 6, wherein said step of selecting a product category comprises:
collecting a polynucleotide sample from the individual;
using a genetic testing cartridge to perform a genotype profiling test and using the result of the test to identify a predisposition of the individual to each of one or more conditions; and
for a given one or combination of the identified predispositions, choosing a product category that can potentially treat the associated condition(s).

20. A method according to claim 19 and comprising using additional information obtained for the individual, for example lifestyle information, in the identification of the condition predispositions and/or the choosing of the product category.

21. A method according to claim 6, wherein said genetic testing cartridge is a disposable single-use cartridge.

22. A point of sale terminal comprising:
access to memory that may be local or remote to the terminal, in which memory there is stored a first database containing a set of product categories and a set of genetic testing cartridge types mapped to the product categories, and a second database containing, for each product category, a set of available products and a set of genotype profiling outcomes mapped to the available products;
an interface for coupling the point of sale terminal to a genetic testing cartridge in order to receive test results from the cartridge;
a graphical user interface for receiving from a terminal user an identification of a product category and for displaying to the user a selected product; and
a processing entity for using the received identification of a product category to lookup said first database to select a genetic testing cartridge type, for receiving a test result via said interface, for using the received test result as a lookup in said second database to select a product, and for causing the graphical user interface to display the selected product to a user.

23. A point of sale terminal according to claim 22, and comprising a holder for retaining a set of genetic testing cartridges belonging to said cartridge types.

24. A point of sale terminal according to claim 23, said processing entity being configured to control said holder to deliver from the holder a test cartridge of said selected test cartridge type.

25. A point of sale terminal according to claim 24, said holder delivering said test cartridge to a position in which it is connected to said interface.

26. A point of sale terminal according to claim 22, said processing means being configured to allow said second database to be updated to reflect available products within a retail premises within which the terminal is located.

27. A point of sale terminal according to claim 22, wherein said memory stores a third database containing a set of condition predispositions and a set of genotype profiling outcomes mapped to said condition predispositions, and said processing entity is configured to receive a test result from a genetic testing cartridge, and use the received result as a lookup to said third database to identify one or more condition predispositions.

28. A point of sale terminal according to claim 27, wherein the processing entity is configured to use the received result as a lookup to said third database in combination with other data obtained for the individual, such as lifestyle information.

29. A point of sale terminal according to claim 27, said processing entity being further configured to display the or each condition predisposition via the graphical user interface, thereby facilitating said selection of a product category suited to treating a condition to which the individual is predisposed.

30. A point of sale terminal according to claim 22, wherein said memory stores a further database containing a set of known metabolisable compounds found in the products in the second database, and said processing entity is configured to receive a user's test result from a genetic testing cartridge, and use the received result to determine the efficacy of one or more of said known metabolisable compounds with respect to the metabolism pathway of the user, then to select a product from the second database based on the determined metabolisable compound efficacies for the user.
